(19) United States
(12) Patent Application Publication
(10) Pub. No.: US 2015/0337290 A1
(43) Pub. Date: Nov. 26, 2015

(54) METHODS AND PRODUCTS FOR MUTATING NUCLEOTIDE SEQUENCES

(71) Applicant: Alexander GUBIN, North Potomac, MD (US)

(72) Inventor: Alexander Gubin, North Potomac, MD (US)

(21) Appl. No.: 14/425,475

(22) PCT Filed: Sep. 5, 2013
(86) PCT No.: PCT/US2013/058236

§ 371 Date: Mar. 3, 2015

Related U.S. Application Data
(60) Provisional application No. 61/698,188, filed on Sep. 7, 2012.

(51) Int. Cl. C12N 15/10; C12N 9/12; C12N 15/11

(52) U.S. Cl. CPC C12N 15/102; C12N 15/11; C12N 9/1241; C12Y 207/07

(57) ABSTRACT

Described herein are products and methods directed to introducing deletions, insertions and substitutions into nucleotide sequences using transposons. Described herein are isolated transposons comprising first and second outside cutter recognition sequences and first and second terminal inverted repeat sequences, wherein the first and second outside cutter restriction recognition sequences are located at least partially internally to the first and second inverted repeat sequences.

### INCORPORATION OF RELATED APPLICATIONS

This application claims the benefit of U.S. Provisional Application No. 61/698,188, filed Sep. 7, 2012, the entirety of which is incorporated herein by reference.

### INCORPORATION OF SEQUENCE LISTING

This application contains a sequence listing submitted electronically via EFS-web, which serves as both the paper copy and the computer readable form (CRF) and consists of a file entitled “075044-8001WO00_seqlist.txt”, which was created on Sep. 5, 2013, which is 4,096 bytes in size, and which is herein incorporated by reference in its entirety.

### FIELD

The field relates to methods and products for mutating nucleotide sequences using transposons.

### SUMMARY

Described herein are isolated transposons comprising first and second outside cutter recognition sequences and first and second terminal inverted repeat sequences, wherein the first and second outside cutter restriction recognition sequences are located at least partially internally to the first and second inverted repeat sequences.

Further described herein are methods for mutating a target nucleotide sequence comprising:

(1) inserting the above transposon into a target nucleotide sequence; and

(2) cleaving outside of the first outside cutter recognition sequence with a first outside cutter enzyme that recognizes the first outside cutter recognition sequence and a second outside cutter enzyme that recognizes the second outside cutter recognition sequence, producing first and second target nucleotide sequence ends, whereby such cleavages, optionally followed by the introduction into and cleavage from the target nucleotide sequences of a first or a first and second nucleotide sequence, result in the deletion of any target nucleotide sequence base pairs duplicated at the transposon introduction site and a deletion, insertion, or substitution of a target nucleotide sequence base pair.

Further described are methods for mutating a target nucleotide sequence comprising the above steps, wherein step (2) comprises:

(a) ligating to the first and second target nucleotide ends a first nucleotide sequence which comprises third and fourth outside cutter recognition sequences that are in reverse orientation,

(b) cleaving the target nucleotide sequence comprising the introduced first nucleotide sequence with a third outside cutter enzyme that recognizes the third outside cutter recognition sequence and cuts outside the third outside cutter recognition sequence and a fourth outside cutter enzyme that recognizes the fourth outside cutter recognition sequence and cuts outside the fourth outside cutter recognition sequence; producing third and fourth target nucleotide sequence ends, wherein the third and fourth target sequence nucleotide ends do not comprise any portion of the first or second inverted repeat sequences or any portion of target nucleotide sequence base pairs duplicated at the transposon introduction site, except for in each case, optionally, overhanging base pairs;

(c) ligating to the third and fourth target nucleotide ends a second nucleotide sequence comprising a base pair to be inserted into the target nucleotide sequence and fifth and sixth outside cutter enzyme recognition sequences that are in reverse orientation

(d) cleaving the target nucleotide sequence comprising the introduced second nucleotide sequence with a fifth outside cutter enzyme that recognizes the fifth outside cutter recognition sequence and cuts outside the fifth outside cutter recognition sequence and a sixth outside cutter enzyme that recognizes the sixth outside cutter recognition sequence and cuts outside the sixth outside cutter recognition sequence; producing fifth and sixth target nucleotide sequence ends and resulting in the target nucleotide sequence comprising a base pair insertion or substitution at the transposon insertion site.

Further described are modified naturally occurring transposons.

Further described are the above methods, wherein the deletion, insertion or substitution comprises a three base pair codon deletion insertion or substitution in target nucleotide sequence.

Further described are the above methods, wherein the target nucleotide sequence is a bacterial plasmid or phage chromosome.

Further described are the above methods, wherein the insertion or substitution comprises the insertion or substitution of a restriction site.

Further described are the above methods, wherein the method is applied to a library of different target nucleotide sequences.

Further described are the above methods, wherein the steps are repeated resulting in the deletion, substitution or insertions located randomly in the target nucleotide sequence.

Further described are the above methods, wherein the first and second target nucleotide sequence ends are selected from the group consisting of compatible, incompatible, symmetric and asymmetric ends.

Further described are the above methods, wherein the first or second target nucleotide end is selected from the group of ends consisting of cohesive, blunt, semi-blunt, 3′ overhanging base pair(s) and 5′ overhanging base pair(s).

Further described are the above methods, wherein, the first nucleotide sequence or the second nucleotide sequence comprise a selectable or screenable marker.

Further described are the above methods, further comprising ligation of the fifth and sixth target nucleotide ends.

Further described are the above methods, wherein the first or second nucleotide ends and the third or forth nucleotide ends are polished prior to their respective ligation.

Further described are the above methods, comprising separating the target nucleotide sequences comprising the inserted transposon, first nucleotide sequence or second nucleotide sequence.

Further described are the above methods, wherein the first and second outside cutter recognition sequences comprise the same base pairs, the third and fourth outside cutter recognition sequences comprise the same base pairs and the fifth and sixth outside cutter recognition sequences comprise the same base pairs.

Further described are the above methods, wherein the first, second, third, fourth, fifth or sixth target nucleotide sequence ends comprises overhanging base pairs.

Further described are the above methods, wherein the second nucleotide sequence comprises three base pairs to be inserted into the target nucleotide sequence and the resulting target nucleotide sequence of step 6 comprises a three base pair substitution or insertion.

Further described are kits comprising the above transposon and a transposase suitable for facilitating a transposition of the transposon into a target nucleotide sequence.

Further described are kits further comprising an outside cutter restriction enzyme that recognizes the first or second outside cutter restriction sites.

Further described are kits comprising a transposase that interacts in a transposition reaction to the inverted repeat sequences of the transposon, an outside cutter enzyme that recognizes the first or second outside cutter restriction sites and optionally a first or second nucleotide sequence, wherein the transposase, first or second nucleotide sequence comprises a mutated base pair to be inserted into a target nucleotide sequence.

### BRIEF DESCRIPTION OF THE DRAWINGS

FIG. 1 depicts schematics 1A-1B, 2A-2D, 3D which schematically represent embodiments showing the insertion of a transposon (1A-B), trim cassette (2A-2D) and mutation cassette and mutations (3A-3D).

FIG. 2 depicts DNA overhangs resulting from cleaving the nucleotide sequence with an outside cutter enzyme(s).

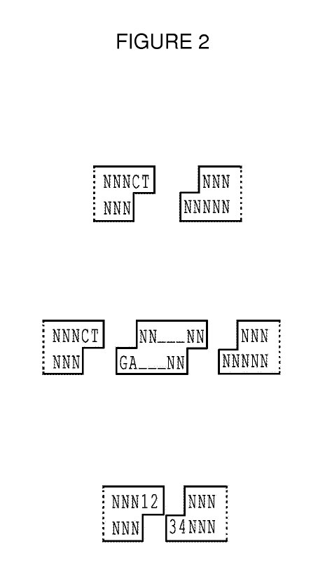

FIG. 3 depicts an agarose gel showing a mutation process (lanes 1-11) as described herein.

### DETAILED DESCRIPTION OF THE INVENTION

The term “isolated” refers to a polypeptide or nucleotide sequence that is removed from at least one component with which it is associated in nature.

The term “nucleotide” as used herein, for example, in the context of a nucleotide sequence or a nucleotide base pair, refers to the chemical meaning of this term as understood to the skilled artisan, including DNA nucleotides or DNA base pairs. As used herein, the term “sequence,” for example, a outside cutter recognition sequence refers to a nucleotide sequence. Generally speaking, as used products and methods herein, nucleotide or sequence refers to DNA nucleotides. However, as used herein, the term “nucleotide sequence” is also meant to encompass nucleotide sequences that contain base pairs that are nucleotide derivatives or analogues.

In an embodiment, a transposon described herein, comprises: (1) two terminal inverted repeat sequences at each transposon termini to interact with a transposase such that the transposase catalyzes the insertion (or transposition) of the transposon into a target nucleotide sequence and (2) and two outside cutter recognition sequence located, respectively, at least partially internally to each of the terminal inverted repeat sequences. Apart from these constituents, it is believed that few if any other additional components are necessary to be included in the transposon sequence.

With respect to the at least partial internal location of the respective outside cutter recognition sequence to its respective terminal inverted repeat sequence, this means that the outside cutter recognition sequence can overlap with (that is, share base pairs with) one or more of the base pairs of its respective terminal inverted repeat sequence. In such an embodiment, the outside cutter recognition sequence comprises one or more base pairs of its respective terminus inverted repeat sequence. In a further embodiment, the outside cutter shares from 1 to 6 base pairs with its respective terminal inverted repeat sequence. In a further embodiment, the outermost base pair of the outside cutter recognition sequence is adjacent to (that is covalently bonded to) (this also may be referred to as directly adjacent to) the innermost base pair of its respective terminal inverted repeat sequence. In a further embodiment, the outermost base pair of the outside cutter recognition sequence is separated by from one to 16 base pairs from the innermost base pair of its respective terminal inverted repeat sequence.

For use in the products and methods described herein, the skilled artisan will desire that the base pair spacing between the respective terminal inverted repeat sequence and respective outside cutter sequence will be designed such that the cleavage by the outside cutter enzyme will be in a desired location, for example, within the respective inverted repeat sequence.

With regard to terminology, as used herein, the description of an outside cutter recognition sequence and its respective terminus inverted repeat sequence corresponds, in embodiments herein, to a first outside cutter recognition sequence and its counterpart first terminal inverted repeat sequence or similarly a second outside cutter recognition sequence and its respective second terminal inverted repeat sequence. Illustrated differently, and purely for the sake of schematic illustration purposes, in embodiments herein, the first outside cutter recognition sequence and first terminal inverted repeat sequence may be visualized as located at the left end (LE) of a transposon viewed in the 5′ to 3′ direction shown below and the second outside cutter recognition sequences and second terminal inverted repeat sequences may be visualized at the right end (RE) of such transposon.

It is understood, however, that this LE and RE designation is arbitrary because of the double stranded nature of a transposon and is therefore used herein solely for the purpose of illustration of the products and methods described herein. It is further understood that the transposon may be contained in a nucleotide cassette or plasmid, such that the LE and RE of the transposon are in fact externally flanked by additional nucleotides, for example, nucleotides of the cassette or plasmid.

Furthermore, using this LE and RE terminology, the first terminal inverted repeat sequence may be referred to as the LE terminal inverted repeat sequence and the second terminal inverted nucleotide sequence may be referred to as the RE terminal inverted nucleotide sequence. Furthermore, the respective outside cutter recognition sequences may be similarly illustrated. That is the first outside cuter recognition sequence being referred to as the LE outside cutter recognition sequence and the second outside cutter recognition sequence being referred to as the RE outside cutter recognition sequence.

As an illustrative example, using the Tn5 “mosaic ends” described herein, a transposon described herein may be as follows:

```
LE RE
5′CTGTCTCTTATACACATCT-CTGCAC[ - - - ]GTGCAG-AGATGTGTATAAGAGACAG
              \hat{ }  \hat{ }
            (SEQ ID NO.: 1) (SEQ ID NO.: 2)
3′GACAGAGAATATGTGTAGA-GACGTG[ - - - ]CACGTC-TCTACACATATTCTCTGTC
          \hat{ }  \hat{ }
            (SEQ ID NO.: 2) (SEQ ID NO.: 1)
```

In the above illustration, the first or LE outside cutter recognition sequence, is adjacent (that is, directly adjacent), internally, to (the innermost base pair of) the LE terminal inverted repeat sequence mosaic end (the covalent bond between the two sequences designated by a dash -) and the RE or second outside cutter recognition sequence is adjacent (that is, directly adjacent), internally, to (the innermost base pair of) the RE terminal inverted repeat sequence mosaic end (also designated by a dash -). In the above illustration, the BsgI outside cutter recognition sequence is used and is placed in reverse orientation on the LE and RE. The ̂ notation above indicates the site of cleavage in this example from the BsgI outside cutter enzyme that recognizes both the first and second outside cutter recognition sequences and in this case introduces a staggered cleavage at 16/14 base pairs external to the most external base pair of the respective outside cutter recognition sequence. In other words, the BsgI outside cutter enzyme produces a staggered cleavage as follows.

BsgI
5′GTGCAG(N)₁₆\hat{ } 3′
3′CACGTC(N)₁₄\hat{ } 5′

The bracket enclosed dashes [---] in the above illustration indicate interior nucleotide sequences in the transposon that can be used to code for desired proteins, for example, selectable or screenable markers or other desirable coding or non-coding nucleotides.

It is further understood with respect to the first and second terminal inverted repeat sequences and first and second outside cutter recognition sequences illustrated in the above embodiments, the transposons embodied herein may or may not also comprise internal to the LE and RE terminal inverted repeat sequences: (1) additional inverted or non-inverted repeat regions (for example the internal inside end (IE) direct repeats of the IS50L and IS50R found in the Tn5 transposon in nature) or (2) additional outside cutter recognition sequences.

With respect to naturally occurring transposons or transposons engineered (or based on) naturally occurring transposons, for use in the methods described herein, the term “transposon” refers to (1) DNA transposons, also referred to in the art as class II transposons, which (2) have terminal inverted repeat regions. However, it is further contemplated that, in alternative embodiments, other mobile genetic elements may be used according to the teachings herein to mutate nucleotide sequences, for example: retrotransposons or DNA transposons having direct (in contrast to inverted) repeat terminal sequences, for example, the IE direct repeat sequence of the IS 50 insertion sequence found in the Tn5 transposon.

Numerous transposons suitable to be engineered for use in the methods and products described herein exist in nature and can be isolated by the skilled artisan using known methods. Bacteriophage (phage) transposons and E. coli transposons are common examples. However, class II transposons have been identified in numerous eukaryotes as well.

Examples of such transposons, that can be used, or modified to be used in the methods described herein include the prokaryotic: Tn3, Tn5, Tn7, Tn9; the bacteriophage Mu transposons; and the eukaryotic Drosophila P and Tcl/Mariner. It is understood by the skilled artisan that the length of the terminal inverted repeat sequences inserted into a target nucleotide as well as the length of the direct repeat target sequence duplication (typically from 2-9 base pairs) caused by certain transposases must be assessed in order to assertion the outside cutter enzyme with an appropriate outside length as well as for the other methods described herein. Such information is known for numerous transposon systems and can be determined using existing methods for transposon systems for which this information is not now known. For example, it is known for the Tn5 engineered mosaic end system, that the inverted repeats are 19 base pairs and the duplicated target nucleotide direct repeats are 9 base pairs.

Moreover, engineered transposons are commercially available and may be used with the methods described herein, when further engineered, for example, to contain outside cutter recognition sequences internal to their inverted repeat termini. Examples of such engineered transposon systems are: the Tn5 transposon based EZ-Tn5 system (Epicentre, Madison Wis., USA); the Mu transposon based Mutation Generation System (Finnzymes Oy, Espoo, Finland); Invitrogen's Mu transposon based Genejumper system; and the Tn7 transposon based GPS system of New England Biolabs.

Accordingly, while isolated naturally occurring transposons may be used herein that naturally contain outside cutter recognition sequences located internally of the respective inverted repeat termini, it is also desirable to engineer naturally occurring transposons in order to insert outside cutter recognition sequences at desired locations internal to inverted repeat sequence termini. Moreover, it is further desirable to engineer naturally occurring transposons so that they have increased efficiency (that is, increased number of transposition events), fidelity (that is, reduced errors), or to include other attributes that are advantageous to the methods described herein, such as, to add selectable or screenable markers to the transposon.

As previously stated, the transposons used in the methods herein, comprise terminal nucleotide inverted repeat sequences. These repeated sequences are located at the respective transposon termini and are termed “inverted” because they are in reverse 5′-3′ orientation. Typically, transposon terminal inverted repeat regions range from 9 to 40 base pairs long. Furthermore, as understood by the skilled artisan, transposon inverted repeat sequences useful for the methods described herein, do not need to be identical. For instance, terminal inverted repeat sequences in naturally occurring transposons can be non-identical and such non-identical inverted repeats are referred to, for example, as imperfect, semi-perfect, or nearly perfect inverted repeats. Example of imperfect inverted repeat sequences are those found in the transposon Tn10 and the transposon Tn7.

Furthermore, terminal inverted repeat regions may be engineered by determining, for example, which sequences are significant for transposition frequency. For instance, specific base pairs in Tn4 terminal inverted repeat sequences have been identified as significant for transposase binding. Zhou, et al., J. Mol. Biol., 276:913-925 (1998). Moreover, 19 base pair Tn4 terminal inverted repeat ends, termed “mosaic ends” have been engineered for increased in vitro transposon frequency. Bhasin, et al., Journal of Biological Chemistry, 274: 37021-37029 (1999). Accordingly, terminal inverted repeat regions may further be engineered, as desired, to incorporate all or a portion of an outside cutter recognition sequence, with the recognition that the skilled artisan would determine whether such mutations to naturally occurring terminal inverted repeat sequences, or to previously engineered of such sequences, would deleteriously affect the transposition reaction in an acceptable fashion for use in the products and methods described herein.

In the transposons described herein, the terminal inverted repeat sequences interact with transposase(s) (generally speaking, one of the roles of the terminal repeat sequences is to be recognized by the transposase(s)), to carry out the transposition (or insertion into a target nucleotide sequence) reaction. In the methods described herein, typically, the transposition reaction will be carried out by transposase(s) which are known to interact in nature with the naturally occurring transposon from which the inverted repeat sequences are taken or derived.

If additional base pairs flank the terminal inverted repeat sequences, these typically, in the methods described herein, will be cleaved off of the transposon prior to its insertion into a target nucleotide sequence. However, the transposon can be designed so that the terminal inverted repeat sequences are externally flanked with, for example, restriction enzyme recognition sequences, which are not cleaved from the transposon and are inserted with the transposon into the target nucleotide sequence.

With regard to the insertion of transposons described herein into target nucleotide sequences, it is understood by the skilled artisan that, depending on the transposon system used, the insertion will be have a given degree of target preference. For example, it has been reported that Tn5 and Mu may have a slight insertion bias towards target sequences containing guanosine (G) and cytidine (C) and Tn7 was found to have significantly less bias but nevertheless to have a small bias towards A/T rich insertion sequences. See Green et al., Mobile DNA, 3:3 (2012). Accordingly, as used herein, the term “random” in the context of target insertion means a small but acceptable degree of target bias, as found for example, in the Tn5 Epicenter commercial system described herein.

With regard to transposases useful in the products and methods described herein, typically, a transposase will be used that is isolated from in vivo transposon reactions for respective transposons. For instance, transposons that catalyze transposition for each of the transposons described herein are known to the skilled artisan.

Moreover, just as isolated naturally occurring transposons and isolated naturally occurring terminal inverted repeat sequences may be engineered by the skilled artisan to meet the purposes of the products and methods described herein, so too, naturally occurring transposases may be engineered by the skilled artisan, for the benefit of the products and methods described herein. For example, naturally occurring transposases may be modified to increase the number of transposition events. For example, a modified Tn5 transposase, having increased transposase activity is described in PCT Patent Application Publication No: WO 98/10077.

Regarding transposition reaction conditions, in vivo conditions have been studied for numerous transposons and it is believed that naturally occurring transposon transposition does no typically require high energy co-factor. With regard to in vitro conditions, suitable reaction conditions and reagents have been published for numerous in vitro transposon systems. For example, for in vitro Tn5 based transposition reactions using, for example, a hyperactive mutant transposase, such as EK54/LP372 Tnp, the following other reaction substituents are described: Mg²⁺, transposon DNA defined by two inverted 19-bp end sequences, and target DNA. For further reagents that may be used with a Tn5 based system see PCT Patent Application Publication No. WO98/10077; Goryshin et al., J. Biol. Chem. 273:7367-7374 (1998).

For further information regarding transposons, transposases and suitable transposition reaction conditions, one is directed to Protein-Nucleic Acid Interactions, Chapter 11 “DNA Transposons,” page 270 et seq. (Royal Society of Chemistry, Bimolecular Science) (Rice et al. Eds) (2008) and Mobile DNA II (Craig) (ASM Press) (Craig et al., Eds) (2002).

As described herein, in certain embodiments, the transposon contains, internally to the respective terminal inverted repeat sequences, outside cutter restriction recognition sequences which are recognized by their counterpart outside cutter enzymes.

An outside cutter enzymes is a restriction endonuclease (RE) that digests a nucleotide sequence outside its recognition sequence(s). Examples of outside cutter restriction enzymes are Type II restriction enzymes of the subclass S, that is, Type IIS restriction enzymes. (also referred to herein as REIIs enzymes or REIIS enzymes). Illustrative examples of outside cutter enzymes are: AcuI, BpuEI, BsgI, BspMI, BsrDI, BtsI, Eco57I, FokI, Ksp632I and MnI I. Further illustrative examples include thermostable Type IIS REs, such as TagII and Tth111II. The recognition sequences and location of cleavage with respect to the recognition sequences for these enzymes are known to the skilled artisan.

Furthermore, additional outside cutter enzymes and their respective recognition sequences are known to the skilled artisan and may be used with the methods and products described herein but, for the sake of conciseness, are each not individually listed herein. Moreover, techniques for identifying and isolating currently undiscovered outside cutter recognition enzymes are known to the skilled artisan, see e.g., Skowron, et al., “A new Thermus sp. class-IIS enzyme sub-family: isolation of a ‘twin’ endonuclease TspDTI with a novel specificity 5′-ATGAA(N(11/9))-3′, related to TspGWI, TagII and Tth111II,” Nucleic Acids Res. 2003 31(14):e74 (2003).

As used herein, the term “outside cutter enzyme” further includes engineered enzymes having outside cutter nuclease activity and recognition sequence specificity. For example, as described in Lippow et al., “Creation of a type IIS restriction endonuclease with a long recognition sequence,” Nucleic Acids Res. 2009 37(9):3061-73 (2009) Additionally, restriction enzymes can be designed by combining restriction enzyme outside cutter nuclease activity with zinc finger recognition specificity. Katada et al., “Artificial restriction DNA cutters as new tools for gene manipulation,” Chembiochem 2510(8):1279-88 (2009).

Moreover, outside cutter enzymes may catalyze either blunt end or staggered base pair cleavage. Furthermore, there is currently no known limit for the number of base pairs outside the recognition sequence(s) that a naturally occurring or engineered outside cutter enzyme may cut. In embodiments herein, the outside cutting range of an outside cutter enzyme starting from the outermost base pair of the recognition sequence to the cleaved base pair is from 1 to 27 base pairs.

Referring now to the Figures, it is first noted that the Figures depict schematic representations and are not drawn to proportional scale regarding, for example, the base pair lengths of the respective nucleotide regions. Furthermore it should be understood that the Figures are for illustrative purposes and are not intended to depict all aspects of the embodiments disclosed elsewhere herein.

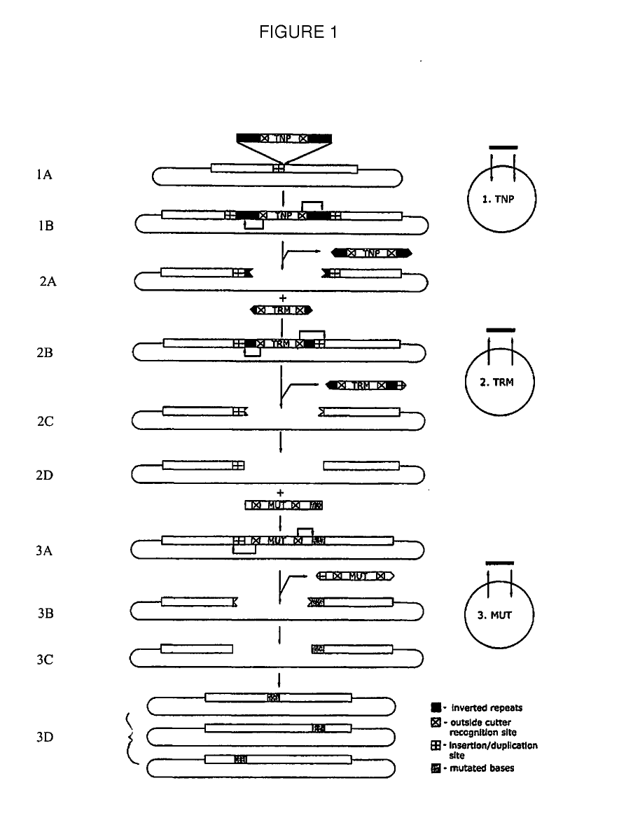

Referring to Schematic 1A of FIG. 1, this schematic depicts an embodiment of the insertion of a transposon nucleotide sequence (denoted “TNP” in the schematics) into an insertion site (denoted as an insertion/duplication site in FIG. 1) located on a target nucleotide sequence. Insertion sites typically will be random sites on target nucleotide sequences (as shown in Schematic 3D of FIG. 1, which shows random mutation sites). In Schematic 1A, the target nucleotide sequence depicted is circular, which is intended to denote that, for example, the target nucleotide sequence may be a plasmid. However, the target nucleotide sequence may also be linear, for example, a chromosome, e.g., a phage chromosome. As shown in Schematic 1A, the transposon contains inverted repeat sequences on its 3′ and 5′ ends and internal to these regions are outside cutter recognition sequences as shown in the schematic).

Referring to Schematic 1B of FIG. 1, this schematic depicts the transposon nucleotide sequence having been inserted into the target nucleotide sequence at an insertion site. Schematic 1B further shows that nucleotides of the target nucleotide sequence at the insertion site have been duplicated at the opposing end of the inserted transposon. Such duplication results, in an embodiment, from the overhang cleavage and DNA polymerase filling ligation that is carried out by certain transposases. Schematic 1B further shows that the outside cutter recognition sequences have been placed in reverse orientation. Schematic 1B further shows that the restriction enzyme cutting is located at a predetermined base pairs. For example, in the embodiment shown in Schematic 1B, the cleavage occurs in the inverted repeat sequences.

Referring to Schematic 2A of FIG. 1, this schematic depicts removal, by cleavage, of a portion of the inserted transposon from the target nucleotide sequence which results in a first pair of target nucleotide sequence ends. The cleavage is carried out, for example, by an outside cutter enzyme (cutting in the orientation shown by opposing arrows shown in Schematic 1B). As shown in the Schematic 2A, a portion of the inverted repeat sequence remains in the target nucleotide sequence. In an embodiment, the precise base pairs of these remaining regions are predetermined based on precise cleavage locations. Such cleavage locations may be provided by, for example, the location of the recognition sequence and the choice of restriction enzyme in the. In Schematic 2A, the ends of the removed transposon portion are shaped as triangles and the counter part ends of the first pair of target nucleotide sequence ends are shaped as triangle cut-outs, to indicate that the ends may be sticky or blunt, depending on the choice of restriction enzyme. Moreover, in a further embodiment, one end may be sticky and the other end may be blunt if different types of restriction enzymes are used to cleave at the opposing ends.

Referring to Schematic 2B of FIG. 1, this schematic depicts the insertion of a first nucleotide sequence, in the embodiment shown, a trimming cassette (denoted “TRM” in the schematics), is inserted into the target nucleotide sequence by ligation of the trimming cassette to the first pair of target nucleotide sequence ends produced by the cleavage depicted in Schematic 2A. As discussed with respect to Schematic 2A above, the restriction enzyme recognitions sites are precisely located so that cleavage occurs at a desired base pair in both orientations. In Schematic 2B, it is shown that portions of the inverted repeat sequences are retained in the target nucleotide sequence and portions of the inverted repeat sequences are introduced in trimming cassette. However, in alternative embodiments, other transposon regions may be retained in the target nucleotide sequence or introduced in the trimming cassette as is desired, for example, spacer regions located between the inverted repeat sequences and the recognition sequences.

Referring to Schematic 2C of FIG. 1, this schematic depicts removal, by cleavage, of the trimming cassette from the target nucleotide sequence and additionally the removal of substantially all of the remaining transposon base pairs from the target nucleotide sequence. In an embodiment, removal of substantially all of the remaining transposon base pairs refers to from 0-5 or 0-10 transposon base pairs remaining in the target nucleotide sequence. Schematic 2C also depicts a second pair of target nucleotide ends produced from this cleavage.

Referring to Schematic 2D of FIG. 1, shows a blunting of the terminal ends of the target nucleotide sequence.

Referring to Schematic 3A of FIG. 1, this schematic depicts the joining of a second nucleotide sequence to the second pair of cleaved target sequence nucleotide ends. In this schematic, the second nucleotide sequence includes mutated nucleotides which are joined to the second pair of target sequence nucleotide ends.

Referring to Schematic 3B of FIG. 1, this schematic depicts the cleaving of the target nucleotide sequence containing the second nucleotide sequence such that a portion of the second nucleotide sequence remains in the target nucleotide sequence and this portion contains at least one mutation relative to the target nucleotide sequence.

Referring to Schematic 3C of FIG. 1, this schematic shows a deletion of base pair(s) of the terminus of the target nucleotide sequence opposite to the terminus containing the at least one mutation and blunt ending of the terminus containing the at least one mutation.

As used herein, the singular form, such as “a transposon” or “the transposon” is intended to include one or more, unless a different meaning is clearly indicated. As used herein, the term “including” means “including but not limited to.” Furthermore, the description of ranges described herein, includes a description of each individual integer between the range, for example, a range of 9-40 would include individually 1, 2, 3 . . . 38, 39, 40.

Further mutation methods using transposons may be found in the following documents, US 2005/0074892 to Lee et al.; WO 2006/017371 to Hansen et al.; and US 2009/0045761 to Jones et al., the entire contents of which are hereby incorporated herein by reference. Furthermore, the contents of all other documents cited herein are hereby incorporated by reference in their entirety.

### EXAMPLES

### Example 1

Materials
Bacterial strain: Escherichia coli TOP10 (Invitrogen, USA),
Plasmid: pUC19,
Transposon: modified Tn5 (Epicentre)
Antibiotics: Carbenicillin, tetracycline, kanamycin,
DNA-related enzymes: Phusion Flash DNA Polymerase, CIAP (Amersham), BsgI, BtsI restriction endonucleases, T4 DNA Polymerase (NE Biolabs, Beverly, Mass., USA), Rapid DNA Ligation Kit (Fermentas), EZ-Tn5™ transposase (Epicentre, Madison, Wis., USA).
DNA purification kits: The isolation of plasmid DNA from bacterial cultures was performed using Zyppy™ Plasmid Miniprep Kit. DNA was isolated from agarose gel using the Zymoclean™ Gel DNA Recovery Kit. DNA was isolated from PCR or restriction digests reactions using DNA Clean & Concentrator™ kit. All kits were supplied by Zymo Research Corporation, Irvine, Calif. USA.

### Methods and Results

Recognition sites for restriction enzymes cleaving outside their recognition sequences are placed internally at the both ends of the transposon preferably immediately adjacent to the transposon ends (“unlocked” transposon).
Antibiotic Resistance Gene (ARG1) is Kan^{R} gene in following examples.
Antibiotic Resistance Gene (ARG2) is Tet^{R} gene in following examples.
Antibiotic Resistance Gene (ARG3) is Amp^{R} gene in following examples.

A general mutagenesis procedure maybe carried out consisting of the following steps:

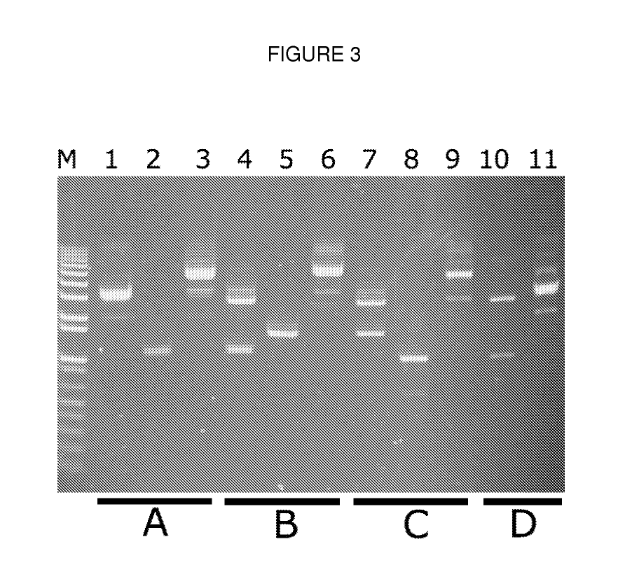

Referring, to FIG. 3:

Step A: Into the target plasmid DNA that can grow in the presence of carbenicillin (Figure, lane 1) the Tn5M (Modified or “unlocked”) transposon (lane 2) is inserted. Bacteria containing a plasmid-integrated Tn5M (lane 3) contain ARG1^{R} gene and can grow in the presence of antibiotic 1 (e.g., kanamycin).

Step B: The plasmid DNA (lane 3) from pooled bacteria is isolated and the majority of transposon is removed by BsgI digestion followed by treatment with Alkaline Phosphatase (lane 4). This DNA is ligated with DNA fragment (lane 5) containing ARG2^{R} gene and appropriately situated BsgI recognition sites and transformed into bacteria. The bacteria containing DNA with integrated ARG2^{R} gene (lane 6) can grow in the presence of antibiotic 2 (e.g., tetracycline).

Step C: The plasmid DNA from pooled bacteria (lane 6) is isolated and all but two bases of the remaining transposon as well as duplicated bases resulted from transposon insertion are removed by BsgI digestion followed by treatment with Alkaline Phosphatase (lane 7). This BsgI digested DNA is ligated with DNA fragment (lane 8) containing ARG1^{R} gene and appropriately situated BsgI recognition sites and transformed into competent bacteria. Bacteria containing plasmid DNA with ARG1^{R} gene (lane 9) can grow in the presence of antibiotic 1 (e.g., kanamycin).

Step D: The plasmid DNA (lane 9) from pooled bacteria is isolated and specified by design number of original bases are removed and specified number of random (or non-random) bases are inserted by BsgI digestion followed by treatment with T4 DNA Polymerase (lane 10). The ends of BsgI digested DNA are intramolecularly joined resulting in the reformation of the target nucleotide sequence with random substitutions, deletions, and/or insertions (lane 11). This DNA is used to transform bacteria. Transformed bacteria are grown on plates supplemented with antibiotic 3 (e.g., carbenicillin). The obtained library is screened or selected for the required properties.

A further discussion of steps A-D is as follows:

Step A: Transposition reaction, transformation into E. coli cells:
Transposition with Tn5M transposon was performed at 37° C. for 2 hr followed by heat inactivation for 10 min at 70° C. The reaction mixture was composed of 1 ul 10× reaction buffer (0.5 M Tris-acetate (PH 7.5), 1.5 M potassium acetate, 100 mM magnesium acetate, and 40 mM spermidine), 200 ng pUC19, 70 ng Tn5M and 1 unit of EZ-Tn5™ transposase (Epicentre, Madison USA) in a total volume 10 ul. The reaction was stopped by the addition of 1 ul stop solution (1% SDS) and incubation at 70° C. for 10 min. Part of the reaction mixture (1-2 ul) was used to transform E. coli TOP10 cells and the cells were plated on LB agar plate containing 50 ug/ul kanamycin to select for cells containing inserted into pUC19 Tn5M transposon. Plasmid DNA was purified from about 10,000 pooled kanamycin resistant colonies.

Step B: Digest of plasmid DNA with BsgI, dephosporylation, ligation, transformation:
Plasmid DNA from Step A (2 ug) was digested in 50 ul reaction by BsgI at 37° C. for 1 hour in NEB 1× reaction buffer 4 (20 mM Tris-acetate, 50 mM potassium acetate, 10 mM Magnesium Acetate, 1 mM Dithiothreitol, pH 7.9@25° C.) in the presence of 80 uM S-adenosylmethionine. After 30 minutes incubation, 5.7 ul of 10× Antarctic Phosphatase Reaction Buffer (50 mM Bis-Tris-Propane-HCl, 1 mM MgCl2, 0.1 mM ZnCl2, pH 6.0@25° C.) and 1 ul of Antarctic Phosphatase (5 units) were added to the reaction and mixed. Reaction was allowed to proceed for 30 more minutes at 37° C. followed by heat inactivation at 65° C. for 20 minutes. This reaction mixture (3.0 ul) was used in ligation with 108 ng of DNA fragment containing Tet^{R} gene in 20 ul reaction in 1× Rapid Ligation Buffer (Fermentas) and 1 ul T4 DNA Ligase (Fermentas) for 60 minutes at room temperature. Part of the reaction mixture (3 ul) was used to transform chemically competent E. coli TOP10 cells and the cells were plated on LB agar plate containing 50 ug/ul tetracycline to select for cells containing inserted DNA containing Tet^{R} gene. Plasmid DNA was purified from about 20,000 pooled tetracycline resistant colonies.

Step C: Digest of plasmid DNA with BsgI, dephosporylation, ligation, transformation:
Plasmid DNA from Step B (2 ug) was digested in 50 ul reaction by BsgI at 37° C. for 1 hour in NEB 1× reaction buffer 4 (20 mM Tris-acetate, 50 mM potassium acetate, 10 mM Magnesium Acetate, 1 mM Dithiothreitol, pH 7.9@25° C.) in the presence of 80 uM S-adenosylmethionine. After 30 minutes incubation, 5.7 ul of 10× Antarctic Phosphatase Reaction Buffer (50 mM Bis-Tris-Propane-HCl, 1 mM MgCl2, 0.1 mM ZnCl2, pH 6.0@25° C.) and 1 ul of Antarctic Phosphatase (5 units) were added to the reaction and mixed. Reaction was allowed to proceed for 30 more minutes at 37° C. followed by heat inactivation at 65° C. for 20 minutes. This reaction mixture (2.5 ul) was used in ligation with 130 ng of DNA fragment containing Kan^{R} gene in 20 ul reaction in 1× Rapid Ligation Buffer (Fermentas) and 1 ul T4 DNA Ligase (Fermentas) for 15 minutes at room temperature. Part of the reaction mixture (3 ul) was used to transform chemically competent E. coli TOP10 cells and the cells were plated on LB agar plate containing 50 ug/ul kanamycin to select for cells with DNA containing Kan^{R} gene. Plasmid DNA was purified from about 20,000 pooled kanamycin resistant colonies.

Step D: Digest of plasmid DNA with BsgI, blunting, recircularization of linear DNA, transformation:
Plasmid DNA from Step C (2 ug) was digested in 50 ul reaction by BsgI at 37° C. for 1 hour in NEB 1× reaction buffer 4 (20 mM Tris-acetate, 50 mM potassium acetate, 10 mM Magnesium Acetate, 1 mM Dithiothreitol, pH 7.9@25° C.) in the presence of 80 uM S-adenosylmethionine. DNA from BsgI digest was purified with Zymo Clean & Concentrator™ Kit and 1 ug was blunted at room temperature in 25 ul reaction in 1× Blunting Buffer (100 mM Tris-HCl, 50 mM NaCl, 10 mM MgCl2, 0.025% Triton X-100, 5 mM dithiothreitol, pH 7.5 at 25° C.), 0.1 mM dNTP Mix and 1 μl of Blunt Enzyme Mix. After 15 minutes enzymes in the blunting reaction were inactivated by heating at 70° C. for 10 minutes. Resulting DNA (10 to 20 ng) was used for intramolecular ligation in 50 ul reaction in 1× Rapid Ligation Buffer and 1 ul T4 DNA Ligase (Fermentas) for 15 minutes at room temperature. Part of the ligation mixture was used to transform chemically competent E. coli TOP10 cells. The cells were plated on LB agar plate containing 50 ug/ul carbenicillin to generate 10,000 to 15,000 independent clones representing variants derived from original DNA plasmid.
In this Example 1 are methods showing how to create a library of variants containing three contiguous nucleotide substitutions at random positions in the pUC19 DNA.

In this example, Steps A to D are performed. In step C DNA fragment containing ARG1^{R} gene is designed to remove three contiguous nucleotides from target DNA and insert three contiguous random nucleotides resulting in random 3 nucleotide substitutions. Sequencing plasmid DNA from independent clones confirmed that 3 nucleotide substitutions occurred randomly in target DNA.

### Example 2

This example shows how to create variants containing 3 contiguous nucleotide deletions with simultaneous 6 contiguous bases insertion (TGGCCA) at random positions in the pUC19 without blunting (polishing) of DNA ends.

The steps in this example are the same as in example 1 with one difference: in step B DNA fragment containing ARG2^{R} gene has differently situated BsgI recognition sites that results in removing introduced Tn5 transposon and all duplicated bases. This change makes unnecessary polishing (blunting) of DNA in step D. Plasmid DNA from Step C is digested with BsgI and DNA ends are intramolecularly joined resulting in the reformation of the target nucleotide sequence with random 3 bases deletions and 6 bases insertion. Presence of newly introduced MscI recognition site (TGGCCA) in plasmid DNA from randomly picked independent clones was confirmed by digestion with MscI.

### Example 3

This Example shows methods for creating variants containing 9 contiguous nucleotide substitutions in the target DNA after TG dinucleotide (any other dinucleotide can be selected if desired).

The steps in this example follow those of Example 1. However, in step C DNA fragment containing ARG1^{R} gene with appropriately situated BsgI recognition sites is modified to limit insertion only after TG in target DNA with subsequent 9 nucleotide substitutions. Sequencing plasmid DNA from several randomly picked clones confirmed that 9 nucleotide substitutions occurred after TGs in target DNA.

1. An isolated transposon comprising first and second outside cutter recognition sequences and terminal first and second inverted repeat sequences, wherein the first and second outside cutter restriction recognition sequences are located at least partially internally to the first and second inverted repeat sequences and wherein the first and second outside cutter recognition sequences are in reverse orientation.

2. A method for mutating a target nucleotide sequence comprising:
(1) inserting the transposon of claim 1 into a target nucleotide sequence; and
(2) cleaving outside of the first outside cutter recognition sequence with a first outside cutter enzyme that recognizes the first outside cutter recognition sequence and a second outside cutter enzyme that recognizes the second outside cutter recognition sequence, producing first and second target nucleotide sequence ends, whereby such cleavages, optionally followed by the introduction into and cleavage from the target nucleotide sequences of a first or a first and second nucleotide sequence, result in the deletion of any target nucleotide sequence base pairs duplicated at the transposon introduction site and a deletion, insertion, or substitution of a target nucleotide sequence base pair.

3. The method of claim 2, wherein step (2) comprises:
(a) ligating to the first and second target nucleotide ends a first nucleotide sequence which comprises third and fourth outside cutter recognition sequences that are in reverse orientation,
(b) cleaving the target nucleotide sequence comprising the introduced first nucleotide sequence with a third outside cutter enzyme that recognizes the third outside cutter recognition sequence and cuts outside the third outside cutter recognition sequence and a fourth outside cutter enzyme that recognizes the fourth outside cutter recognition sequence and cuts outside the fourth outside cutter recognition sequence; producing third and fourth target nucleotide sequence ends, wherein the third and fourth target sequence nucleotide ends do not comprise any portion of the first or second inverted repeat sequences or any portion of target nucleotide sequence base pairs duplicated at the transposon introduction site, except for in each case, optionally, overhanging base pairs;
(c) ligating to the third and fourth target nucleotide ends a second nucleotide sequence comprising a base pair to be inserted into the target nucleotide sequence and fifth and sixth outside cutter enzyme recognition sequences that are in reverse orientation
(d) cleaving the target nucleotide sequence comprising the introduced second nucleotide sequence with a fifth outside cutter enzyme that recognizes the fifth outside cutter recognition sequence and cuts outside the fifth outside cutter recognition sequence and a sixth outside cutter enzyme that recognizes the sixth outside cutter recognition sequence and cuts outside the sixth outside cutter recognition sequence; producing fifth and sixth target nucleotide sequence ends and resulting in the target nucleotide sequence comprising a base pair insertion or substitution at the transposon insertion site.

4. The transposon of claim 1, wherein the transposon is not naturally occurring.

5. The method of claim 2, wherein the deletion, insertion or substitution comprises a three base pair codon deletion insertion or substitution in target nucleotide sequence.

6. The method of claim 2, wherein the target nucleotide sequence of claim 1 is a bacterial plasmid or phage chromosome.

7. The method of claim 2, wherein the insertion or substitution comprises the insertion or substitution of a restriction site.

8. The method of claim 2, wherein the method is applied to a library of different target nucleotide sequences.

9. The method of claim 2, wherein the steps are repeated resulting in the deletion, substitution or insertions located randomly in the target nucleotide sequence.

10. The method of claim 2, wherein the first and second target nucleotide sequence ends are selected from the group consisting of compatible, incompatible, symmetric and asymmetric ends.

11. The method of claim 2 wherein the first or second target nucleotide end is selected from the group of ends consisting of cohesive, blunt, semi-blunt, 3′ overhanging basepair(s) and 5′ overhanging basepair(s).

12. The method of claim 2, wherein the transposon, first nucleotide sequence or the second nucleotide sequence comprise a selectable or screenable marker.

13. The method of claim 3, further comprising ligation of the fifth and sixth target nucleotide ends.

14. The method of claim 3, wherein the first or second nucleotide ends and the third or forth nucleotide ends are polished prior to their respective ligation.

15. The method of claim 3, comprising separating the target nucleotide sequences comprising the inserted transposon, first nucleotide sequence or second nucleotide sequence.

16. The method of claim 3, wherein the first and second outside cutter recognition sites comprise the same base pairs, the third and fourth outside cutter recognition sites comprise the same base pairs and the fifth and sixth outside cutter recognition sites comprise the same base pairs.

17. The method of claim 3, wherein the first, second, third, fourth, fifth or sixth target nucleotide sequence ends comprises overhanging base pairs.

18. The method of claim 3, wherein the second nucleotide sequence comprises three base pairs to be inserted into the target nucleotide sequence and the resulting target nucleotide sequence of step 6 comprises a three base pair substitution or insertion.

19. A kit comprising the transposon of claim 1.

20. The kit of claim 18, further comprising a transposase that binds to the inverted repeats regions of the transposon, an outside cutter restriction enzyme that recognizes the first or second outside cutter restriction sites and optionally a first or second nucleotide sequence, wherein the transposase, first or second nucleotide sequence comprises a mutated base pair to be inserted into a target nucleotide sequence.


SEQUENCE LISTING (2 sequences)

SEQ ID NO: 1 - DNA, 25 bp, Artificial Sequence
  Description of Artificial Sequence Example restriction enzyme recognition sequence
ctgtctcttatacacatctctgcac

SEQ ID NO: 2 - DNA, 25 bp, Artificial Sequence
  Description of Artificial Sequence Example restriction enzyme recognition sequence
gtgcagagatgtgtataagagacag